(19) United States
(12) Patent Application Publication
(10) Pub. No.: US 2005/0239056 A1
(43) Pub. Date: Oct. 27, 2005

(54) Methods and kits for predicting an infectious disease state

(76) Inventors: Roger N. Piasio, Cumberland, ME (US); Howard Faden, Buffalo, NY (US)

Correspondence Address: T. D. FOSTER, 12760 HIGH BLUFF DRIVE, SUITE 300, SAN DIEGO, CA, 92130, US

(21) Appl. No.: 10/989,973

(22) Filed: Nov. 16, 2004

Related U.S. Application Data
(60) Provisional application No. 60/525,411, filed on Nov. 26, 2003.

(30) Foreign Application Priority Data
Nov. 26, 2003 (WO) PCT/US03/37787

(51) Int. Cl. C12Q 1/70; G01N 33/554; G01N 33/569

(52) U.S. Cl. 435/5; 435/7.32

(57) ABSTRACT

The present invention discloses methods for predicting an infectious disease state of a subject. The methods are rapid, simple, and do not require culturing of the causative infectious agent.

The present application claims benefit of priority to the following application, which is incorporated by reference herein in its entirety: U.S. Provisional Patent Application No. 60/525,411, entitled “Methods and Kits for Predicting an Infectious Disease State”, filed on Nov. 26, 2003.

### TECHNICAL FIELD

The present invention relates generally to the area of medicine, and more particularly concerns methods for predicting an infectious disease state of a subject.

### BACKGROUND OF THE INVENTION

Colonization of the nasopharynx by pathogens occurs in humans of all ages and often accompanies or precedes pathogen-caused disease (Smart et al. (1987) Epidem. Infect., 98:203-209). Pathogens include bacterial, viral, and eukaryotic pathogens. These diseases include respiratory tract infections such as pneumonia (including atypical pneumonia), bronchitis, sinusitis, and influenza or influenza-like illnesses, as well as diseases not limited to the respiratory tract, including middle ear infections (such as otitis media) and conjunctivitis. A disease caused by a pathogen (for example, a respiratory infection) can also share symptoms with a disease not primarily caused by an infectious organism (for example, chronic obstructive pulmonary disease, or COPD), and a diagnostic differential is frequently needed in order for a physician to recommend appropriate therapy.

Otherwise healthy persons who harbor nasopharyngeal colonies of pathogens have generally lower counts of pathogenic bacteria than do individuals who are both colonized and suffering from a disease caused by the same pathogen. There is a need for at least semiquantitative information regarding the level of pathogens in the nasopharynx of an individual, permitting a physician to distinguish a carrier (healthy, but colonized individual) from a colonized individual who also suffers from a disease caused by the pathogen in question. Such information is especially important when a disease is occurring at a high incidence or at epidemic levels, or where a pathogen is widespread but not all carriers are diseased (for example, in day care centers or preschools where young children are virtually all colonized by pathogens that can cause acute otitis media, or in senior residences or nursing homes where many residents can be colonized by pathogens that cause pneumonia). Such information is also valuable when symptoms of a disease caused by one pathogen are identical or similar with symptoms of a disease not caused by that pathogen, for example, distinguishing a bacterial respiratory infection from a viral respiratory infection or from a respiratory illness not caused by any pathogen. It is desirable for a physician to be able to rapidly and accurately distinguish a healthy individual who may be colonized by a pathogen, from an individual who suffers from a disease caused by that pathogen, in order to correctly prescribe the necessary therapy (for example, the appropriate antibiotic or antibiologic).

Culturing for pathogens that cause respiratory tract infections or that are believed to colonize the nasopharynx, followed by identification by microscopic or biochemical criteria, remains the “gold standard” for establishing the presence or absence of a pathogen. However, culturing is time consuming (generally taking periods of from about 3 to about 7 days), and both culturing and the subsequent microscopic or biochemical identification tests require highly skilled personnel. Culturing is a sensitive method of detection, able in some instances to detect a single pathogen cell, but is highly susceptible to contamination. For example, a culture can be contaminated by species other than the pathogen of interest, for example, by bacteria that are part of the normal nasopharyngeal flora, which may overgrow the culture and obscure the results. Other detection methods rely on nucleic acid amplification. While generally faster than culturing, nucleic acid amplification methods are again highly sensitive (able to detect a single copy of a specific nucleic acid sequence), and thus susceptible to contamination. The sensitivity of culturing or nucleic acid detection can result in “false positives” when the subject is a “carrier” or an individual who is colonized by the pathogen in question but who is healthy, that is to say, who has no symptoms of disease caused by that pathogen. Both culturing and nucleic acid amplification methods require specialized equipment and trained technicians. Neither culturing nor nucleic acid amplification are appropriate technologies for a rapid, point-of-care diagnostic, such as a test that can be carried out in a physician's office or at a patient's bedside.

Diagnostic uncertainty was identified in a survey of physicians to be a leading factor in the prescription of antibiotics (1998 Massachusetts Physician Survey, Alliance for the Prudent Use of Antibiotics, available at www.tufts.edu/med/apua/Research/physicianSurvey1-01/physicianSurvey.htm). Even though the same physicians surveyed cited their concerns of antibiotic resistance as the most important motivation to not prescribe antibiotic use, diagnostic uncertainty was again the main reason for their decisions to prescribe broad-spectrum rather than narrow-spectrum antibiotics, typical of the inappropriate use of antibiotics (“Antibiotic Resistance: Synthesis of Recommendation by Expert Policy Groups”, Avorn et al (editors), Alliance for the Prudent Use of Antibiotics and the World Health Organization, 2001, 155 pp.). Thus, there is an urgent need for methods to predict with high certainty if a patient is not in need of antibiotic therapy, or if necessary antibiotic therapy should be narrow-spectrum rather than broad-spectrum.

The present invention provides methods that answer the need for a rapid and accurate method to distinguish a healthy individual who may be colonized by a pathogen, from an individual who suffers from a disease caused by that pathogen. The methods can be of use in determining whether or not a subject should be treated with an antibiotic or antibiologic, and in determining the appropriate type of antibiotic or antibiologic. Such a method is preferably inexpensive, technically simple, and is preferably a point-of-care diagnostic that allows a physician to make an immediate treatment decision.

### DESCRIPTION OF THE INVENTION

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well known and commonly employed in the art. Where there are discrepancies in terms and definitions used in references that are incorporated by reference, the terms used in this application shall have the definitions given herein. Other technical terms used herein have their ordinary meaning in the art that they are used, as exemplified by a variety of technical dictionaries (for example, Chambers Dictionary of Science and Technology, Peter M. B. Walker (editor), Chambers Harrap Publishers, Ltd., Edinburgh, UK, 1999, 1325 pp.). The inventors do not intend to be limited to a mechanism or mode of action. Reference thereto is provided for illustrative purposes only.

The present invention includes a method of rapidly determining the disease state of a subject, wherein the disease is caused by an infectious organism, including the steps of: (a) providing a nasopharyngeally derived sample from the subject; (b) contacting a binding agent directly to the sample, wherein the binding agent is capable of specifically binding to an epitope derived from the infectious organism; (c) allowing the binding agent to specifically bind to and form a complex with the epitope derived from the infectious organism present in the sample; and (d) detecting the complex, wherein the detection is positive if concentration of the infectious organism in the sample is greater than or equal to than a reference concentration, and the detection is negative if concentration of the infectious organism in the sample is less than the reference concentration.

The method of the present invention may be applied to any subject who is suspected of having a disease caused by an infectious organism, where the infectious organism can be at least potentially found in the nasopharyngeal area of the diseased person, or who has disease symptoms for which a diagnostic differential is necessary for appropriate therapy. Such subjects are preferably human subjects, including infants, children, and adult humans of any age.

The method of the present invention may be applied to any disease caused by an infectious organism, where the infectious organism can be at least potentially found in the nasopharyngeal area of the diseased person. Diseases that are of particular relevance include respiratory tract infections (such as, but are not limited to, influenza-like illnesses, pneumonia, bronchitis, and sinusitis), as well as non-respiratory tract infectious diseases (such as, but not limited to, acute otitis media and conjunctivitis). The infectious organism can be any infectious organism that potentially occurs in the nasopharyngeal area and that can cause an infectious disease of interest, or that can cause disease symptoms for which a diagnostic differential is necessary for appropriate therapy. Infectious organisms of interest include pathogenic bacteria (including mycoplasmas), pathogenic viruses, and eukaryotic pathogens (including fungi and protozoans). Infectious organisms that are commonly present in the nasopharynx and oropharynx of healthy humans, and that are at least potentially pathogenic, include bacteria such as Acinetobacter species, viridans streptococci, beta-hemolytic streptococci (including Group A beta-hemolytic streptococci such as Streptococcus pyogenes), non-hemolytic streptococci, Streptococcus pneumoniae, staphylococci (including coagulase-negative staphylococci and Staphylococcus aureus), micrococci, Corynebacterium species (including Corynebacterium diphtheriae), Neisseria species (including Neisseria meningitidis and Neisseria gonorrhoeae), Cryptococcus neoformans, Mycoplasma species, Haemophilus influenzae, Haemophilus parainfluenzae, Moraxella (Branhamella) catarrhalis, enterobacteria, Lactobacillus species, Veillonella species, Mycobacterium species, Pseudomonas species, Klebsiella species (including Klebsiella ozaenae or Klebsiella pneumoniae), Eikenella corrodens, Bacteroides species, Peptostreptococcus species, Actinomyces species, and spirochaetes; fungi, such as Candida albicans and filamentous fungi; and viruses, such as herpes simplex virus (“Bailey and Scott's Diagnostic Microbiology”, 9th edition, Baron et al. (editors), Mosby, St. Louis, Mo., 1994, pp. 220ff.).

One step of the method includes providing a nasopharyngeally derived sample from the subject. Any suitable nasopharyngeally derived sample may be used. Preferred nasopharyngeally derived samples include, but are not limited to, a nasopharyngeal swab, a nasopharyngeal wash, a nasopharyngeal discharge, a nasopharyngeal aspirate, a nasal swab, a nasal wash, a nasal discharge, a nasal aspirate, and combinations thereof. For use in the method of the present invention, suitable nasopharyngeally derived samples may need minimal preparation (for example, collection into a suitable container), or more extensive preparation (such as, but not limited to, removal, inactivation, or blocking of undesirable material, such as contaminants, undesired cells or cellular material, or endogenous enzymes; treatment with buffers or chemical reagents; filtration, centrifugation, size selection, or affinity purification; cell fixation, permeabilization, or lysis; and concentration or dilution). In one non-limiting example, the epitope of interest is a soluble carbohydrate that is released from Streptococcus pneumoniae by treatment with a lysing agent (for example, a buffer containing detergents or surfactants).

Another step of the method includes contacting a binding agent directly to the sample, wherein the binding agent is capable of specifically binding to an epitope derived from the infectious organism. The epitope derived from the infectious organism can be any suitable epitope, including, but not limited to, peptides, polypeptides, proteins, glycoproteins, carbohydrates, lipids, glycolipids, lipoproteins, nucleic acids, antigens, enzymes, receptors, cell wall components, whole cells of the infectious organism, fragments of the infectious organism, substances (such as toxins, enzymes, and exopolymers) secreted by the infectious organism, and combinations thereof. The epitope can optionally be modified, for example, by physical or chemical modification; the binding agent can be capable of specifically binding to the modified epitope. Modification of the epitope can include any suitable modification, including, but not limited to, treatment with chemical reagents or enzymes, oxidation or reduction, labelling with a detectable label, and covalent or non-covalent attachment of the epitope to a separate moiety, molecule, molecular structure, or surface. The binding agent may be capable of binding to a mimotope, such as a peptide, that mimics an epitope naturally derived from the infectious organism (see, for example, Kieber-Emmons (1998) Immunol. Res., 17:95-108; Shin et al. (2001) Infect. Immun., 69:3335-3342; Beenhouwer et al. (2002) J. Immunol., 169:6992-6999; Hou and Gu (2003) J. Immunol., 170:4373-4379; and Tang et al (2003) Clin. Diagn. Lab. Immunol., 10:1078-1084, which are incorporated by reference in their entirety herein). The binding agent is contacted directly to the sample, which may have undergone prior minimal or more extensive preparation, but which has not been subjected to culturing or nucleic acid amplification for the infectious organism.

Binding agents can be virtually any molecule or combination of molecules capable of recognizing and binding the epitope. Such binding agents can include, without limitation, peptides, polypeptides, antibodies, Fab fragments, fusion proteins, chimeric molecules, nucleic acids, nucleic acid mimics (for example, peptide nucleic acids), cell surface antigens, carbohydrates, or combinations thereof. In one preferred embodiment, the binding agent includes an antibody (monoclonal or polyclonal, natural, modified, or recombinant) or an antibody fragment (such as an Fab fragment or single-chain antibody variable region fragment); methods of preparing, modifying, and using such antibodies or antibody fragments are known in the art (see, for example, “Antibodies: A Laboratory Manual”, E. Harlow and D. Lane, editors, Cold Spring Harbor Laboratory, 1988, 726 pp; “Monoclonal Antibodies: A Practical Approach”, P. Shepherd and C. Dean, editors, Oxford University Press, 2000, 479 pp.; and “Chicken Egg Yolk Antibodies, Production and Application: IgY-Technology (Springer Lab Manual)”, by R Schade et al., editors, Springer-Verlag, 2001, 255 pp., which are incorporated by reference in their entirety herein). The binding agent can include an antigen, such as an antigen capable of specifically binding to an antibody that recognizes an epitope derived from the infectious organism. In other embodiments, the binding agent can include a nucleic acid or nucleic acid mimic aptamer that binds a target such as a peptide or small molecule, or a receptor that binds a ligand, or a ligand that binds a receptor.

The binding agent can optionally include a functional group (such as a chemically reactive moiety or cross-linking moiety) or a detectable label; methods to introduce such functional groups or detectable labels are known in the art (see, for example, R. P. Haugland, “Handbook of Fluorescent Probes and Research Products”, 9^{th} edition, J. Gregory (editor), Molecular Probes, Inc., Eugene, Oreg., USA, 2002, 966 pp.; Seitz and Kohler (2001), Chemistry, 7:3911-3925; Pierce Technical Handbook, Pierce Biotechnology, Inc., 1994, Rockford, Ill.; and Pierce 2003-2004 Applications Handbook and Catalog, Pierce Biotechnology, Inc., 2003, Rockford, Ill., which are incorporated by reference in their entirety herein). The binding agent may be free in solution, or may be temporarily or permanently affixed onto a separate moiety, molecule, molecular structure, or surface. In one non-limiting example, the binding agent can be temporarily immobilized by drying onto a surface, wherein addition of a fluid can cause the binding agent to become mobile. In another non-limiting example, the binding agent can be permanently immobilized by covalent or non-covalent attachment to a surface, such as to a membrane, microplate well, tube, chip, or slide.

In one preferred embodiment, the binding agent binds monovalently to the epitope of interest. In another preferred embodiment, the binding agent binds multivalently, for example bivalently and optionally bispecifically, to the epitope (or mimotope) of interest. The binding agent can be used in more than one form or type, for example, where the binding agent is an antibody or antibody fragment and is used in a sandwich assay that involves a binding agent to immobilize the epitope and a detectably labelled binding agent that binds the same epitope.

The binding agent's ability to specifically bind to an epitope derived from the infectious organism can be improved by means known in the art, for example, by selection of a peptide-sequence based on panning methods (see, for example, Coomber (2001) Methods Mol. Biol, 178:133-145; Zhou et al. (2002) Proc. Natl. Acad. Sci. USA, 99:5241-5246; Fehrsen and du Plessis (1999) Immunotechnology, 4:175-184; Deng et al. (1994) J. Biol. Chem., 269:9533-9538; Burioni et al. (1998) Res. Virol., 149:327-330; Boel et al (1998) Infect. Immun., 66:83-88; and Parsons et al. (1996) Protein Eng., 9:1043-1049, which are incorporated by reference in their entirety herein).

Improvement of the binding agent's ability to bind to an epitope derived from the infectious organism (or to a mimotope mimicking the epitope) can use display methods as are known in the art, including displaying on a polypeptide (Kamb, et al, U.S. Pat. No. 6,025,485; Christmann et al., 1999, Protein Eng., 12:797; Abedi et al, 1998, NucleicAcids Res., 26:623; Peelle et al., 2001, J Protein Chem., 20:507), a phage (He, 1999, J. Immunol. Methods, 231: 105; Smith, 1985, Science, 228:1315), a ribosome (Schaffitzel et al., 1999, J. Immunol. Methods, 231:119; Roberts, 1999, Curr. Opin Chem. Biol., 3:268), an mRNA (Wilson et al., 2001, Proc. Natl. Acad. Sci., 98:3750), or a yeast cell surface (Yeung and Wittrup, 2002, Biotechnol. Prog., 18:212; Shusta et al., 1999, J. Mol. Biol., 292:949), a bacterial cell surface (Leenhouts et al., 1999, Antonie Van Leeuwenhoek, 76:367; Christmann et al., 2001, J. Immunol. Methods, 257:163), or a bacterial spore surface (Wittrup, 2001, Curr. Opin. Biotechnol., 12:395; Boder and Wittrup, 1998, Biotechnol. Prog., 14:55). All references cited in this paragraph are incorporated by reference in their entirety herein.

Another step of the method includes allowing the binding agent to specifically bind to and form a complex with the epitope derived from the infectious organism present in the sample. The binding of the binding agent to the epitope can be by any suitable means, including, but not limited to, covalent binding, non-covalent binding, antibody-antigen recognition, receptor-ligand binding, aptamer-nucleic acid binding, physical adsorption, electrostatic forces, ionic interactions, hydrogen bonding, hydrophilic-hydrophobic interactions, van der Waals forces, magnetic forces, and combinations thereof. Preferably, the binding agent binds to the epitope with sufficient specificity to give minimal or no non-specific or cross-reactive binding between the binding agent and an epitope derived from sources other than the infectious organism of interest (such as from cells or tissues of the human subject, or from other infectious or non-infectious species). The specific binding of the binding agent to the epitope preferably results in a complex of sufficient stability to be detected.

Another step of the method includes detecting the complex, wherein the detection is positive if concentration of the infectious organism in the sample is greater than or equal to a reference concentration, and the detection is negative if concentration of the infectious organism in the sample is less than the reference concentration. Detection of the complex can be direct, such as by detection of a label on the binding agent. Alternatively, detection of the complex can be indirect, by any suitable means, including, but not limited to, the use of a secondary antibody, such as a secondary antibody bearing a detectable label. Useful detectable labels include, but are not limited to, fluorophores, luminophores, members of resonance energy transfer pairs, lanthanides, dyes, pigments, radioactive isotopes, magnetic labels, spin labels, heavy atoms, metals, particles (such as gold particles or magnetic particles), and enzymes.

Detection of the complex is positive if the concentration of the infectious organism in the nasopharyngeally derived sample is greater than or equal to a reference concentration. Conversely, detection of the complex is negative if the concentration of the infectious organism in the nasopharyngeally derived sample is less than the reference concentration. The reference concentration selected for a given infectious organism depends on several factors, including, but not limited to, the nature of the binding agent and of the epitope derived from the infectious organism, the type of nasopharyngeally derived sample, and the type of subject (for example, an adult or a child). Reference concentrations can be established by routine testing. Detection can be linear (such as spectrophotometric measurement of product formation by an enzymatic reaction) or non-linear (such as visual detection of a gold label). Detection is optionally at least semi-quantitative, for example, judged to be greater than or equal to, or less than, a reference value. Detection can be optionally quantitative, wherein a positive detection signal can be correlated to a range of concentrations of the infectious organism.

Subjects may be nasopharyngeal “carriers” of an infectious organism, that is to say, otherwise healthy but nasopharyngeally colonized, generally at relatively lower concentrations, by the infectious organism, where a relatively higher concentration of the infectious organism is associated with symptoms of a disease caused by that organism. In such a case, a desirable reference concentration is a concentration below which a nasopharyngeally derived sample from a subject who either is not colonized by the infectious organism in question, or who is colonized by the infectious organism but otherwise healthy, gives a negative detection result. This same reference concentration is preferably a concentration at or above which a nasopharyngeally derived sample from a subject who is colonized and diseased by the infectious organism gives a positive detection result.

Thus, in one embodiment of the invention, a positive detection result indicates that the subject is at least colonized by the infectious organism of interest, or is colonized and diseased by that organism. In one alternative embodiment of the invention, a negative detection result preferably indicates that the subject is not colonized by the infectious organism of interest to a level associated with a disease caused by that infectious organism.

A desirable reference concentration preferably yields a positive predictive value (that is to say, the probability that the subject with a positive detection result is diseased by the infectious organism) of at least about 80%, more preferably of at least about 90%, and most preferably of at least about 95%. A desirable reference concentration preferably yields a negative predictive value (that is to say, the probability that the subject with a negative detection result is not diseased by the infectious organism) of at least about 80%, more preferably of at least about 90%, and most preferably of at least about 95%.

The method of the invention may be carried out by means of a suitable assay. Non-limiting examples of suitable assays for performing the method include dipstick or test strip assays, flow-through assays, chromatographic assays, affinity separation assays, lateral flow assays, latex agglutination assays, radioimmunometric assays, enzyme-linked immunosorbent assays, fluorescence assays, and luminescence assays. Assays can be run in any suitable format, including, but not limited to, membranes, filters, microtiter plates, tubes, chips, slides, and flow-through chambers. Preferably, the assay is rapid, most preferably sufficiently rapid to produce results within a relatively brief period of time, such as the time of a subject's consultation with a physician or other health-care provider.

Kits can be designed for convenience in performing the method, according to the assay used. Kits can include, in addition to a means for performing the assay, means for collecting and appropriately treating a nasopharyngeally derived sample (such as a swab, a means to aspirate a sample, wash solutions or buffers, chemical or enzymatic reagents, filters, centrifuge tubes, and the like). Kits can include materials (such as gloves and other personal safety equipment, biohazard disposal containers, or decontamination materials) that aid in the safe handling of potentially hazardous samples. Kits can include instructions for the use of the kit, for example, instructions in the form of a brochure, leaflet, pamphlet, booklet, or audiovisual materials.

A non-limiting example of a method of the present invention and kit for performing the method follows. This example includes a method for rapidly determining whether a subject is free of an infectious disease, such as acute otitis media, caused by Streptococcus pneumoniae, including the steps of: (a) providing a nasopharyngeally derived sample (such as a nasopharyngeal swab or nasopharyngeal wash or nasal wash) from the subject; (b) contacting a binding agent that includes an antibody capable of specifically binding to an epitope consisting of a soluble cell wall polysaccharide antigen present on all clinical strains of S. pneumoniae; (c) allowing the binding agent (antibody) to bind to the epitope (S. pneumoniae antigen) and form a complex; (d) detecting the complex, wherein positive detection occurs when the concentration of S. pneumoniae is greater than or equal to a reference concentration of 1×10⁴ colony-forming units per milliliter and is manifested as a visible colored signal, and negative detection occurs when the concentration of S. pneumoniae is less that a reference concentration of 1×10⁴ colony-forming units per milliliter and is manifested by the absence of a visible colored signal.

The applicants' assignee, Binax, Inc., introduced to the market in 1999 its NOW® immunochromatographic (“ICT”) rapid diagnostic test kit for the detection of the cell wall polysaccharide antigen common to all serogroups of Streptococcus pneumoniae. This test is approved by the United States Food and Drug Administration for detecting the antigen in urine samples, and is described in U.S. Pat. No. 5,877,028, U.S. patent application Ser. No. 09/156,486, and U.S. patent application Ser. No. 09/518,165, and in U.S. patent application Ser. No. 09/397,110 which discloses the efficacy of the test in detecting the target antigen in samples of other human bodily fluids in addition to urine (all of these patent and patent applications are incorporated by reference in their entirety herein). The test has the capability to detect concentrations of S. pneumoniae greater than 1×10⁴ colony-forming units (CFU) per milliliter of sample (urine or other fluids). It can be run in 15 minutes by anyone capable of reading and comprehending the simple directions provided with the test kits as sold. The test requires no special equipment and can be run at any patient site, such as in a physician's clinic or at a patient's bedside, and is thus an appropriate test for point-of-care health services.

It has been found by applicant's assignee and by others (Faden et al. (2002), Pediatr. Infect. Dis. J, 21:791-792) that children who are nasopharyngeally colonized with S. pneumoniae excrete the cell wall polysaccharide antigen at a relatively high rate, and that otherwise healthy but colonized children tend to give the same positive urine test results as are obtained with urine from children who are both colonized and diseased. Pending, co-assigned U.S. patent application Ser. No. 10/083,476, which is incorporated by reference in its entirety herein, describes methodology for modifying the test to diminish the incidence of false positives obtained with healthy carrier children.

Acute otitis media (AOM), a common childhood disease, is most often caused by bacterial infection of the middle ear (see, for example, Del Beccaro et al (1992), J. Pediatrics, 120:81-84; Harper, M. B. (1999), Pediatr. Infect. Dis. J, 18:1120-1124; and Klein, J. O. (1994) Clin. Infect. Dis., 19:823-833, which are incorporated by reference in their entirety herein). In developed countries such as the United States, the three pathogens responsible for most bacterial AOM are Streptococcus pneumoniae, non-typable Haemophilus influenzae, and Moraxella catarrhalis, all of which are also respiratory pathogens. Simultaneous cultures from the nasopharynx and from the middle ear have been shown to be strongly correlated (see, for example, Howie and Ploussard (1971) Pediatr. Dig., 31-35; Kamme et al. (1971) Scand. J. Infect. Dis., 3:217-225; Schwartz et al. (1979) J. Am. Med Assoc., 241:2170-2173; and Faden et al. (1990) Pediatr. Infect. Dis. J., 9:623-626, which are incorporated by reference in their entirety herein).

It has been shown that both carriage and quantity of Streptococcus pneumoniae, non-typable Haemophilus influenzae, and Moraxella catarrhalis increased during active otitis media episodes compared with healthy periods in children. At the same time, the nonpathogenic flora of the nasopharynx decreased in carriage, suggesting that respiratory pathogens become relatively more important in the nasopharyngeal environment during periods of active otitis media disease (Faden et al. (1990) Pediatr. Infect. Dis. J., 9:623-6260). Colonized, diseased children have greater nasopharyngeal concentrations of Streptococcus pneumoniae than do similarly colonized, but otherwise healthy children (carriers). When the NOW® ICT Test is used to test liquid nasopharyngeal samples from colonized but healthy children (carriers) and from colonize, diseased children, a semiquantitative and clinically useful difference in test results is observed. The NOW® ICT Test has the advantages over culturing and nucleic acid amplification methods of being rapid and technically simple to run, and thus appropriate as a point-of-care diagnostic.

According to the present invention, the NOW® Streptococcus pneumoniae ICT Test can be used on nasopharyngeally derived samples to distinguish between otherwise healthy subjects colonized by S. pneumoniae from subjects who are colonized and diseased by S. pneumoniae. Repeated test results from many sources, conducted with the NOW® Streptococcus pneumoniae ICT Test as presently sold, have demonstrated that colonized children whose nasopharyngeal samples are positive for S. pneumoniae by culture, but yield a negative result in the NOW® ICT Test, are free of disease caused by S. pneumoniae. Thus, the NOW® Streptococcus pneumoniae ICT Test performed on a nasopharyngeal sample is a reliable indicator of good negative predictive value that the subject from whom the sample was obtained is free of disease caused by S. pneumoniae, and therefore is not in need of antibiotic therapy directed at S. pneumoniae infections.

A positive nasopharyngeal culture with Streptococcus pneumoniae has been reported to have little positive predictive value for S. pneumoniae's presence in the middle ear (Faden et al. (1990) Pediatr. Infect. Dis. J, 9:623-626 and Gehanno et al. (1996) Pediatr. Infect. Disease J., 15:329-332). However, it has been shown that nasopharyngeal cultures have a high (greater than 95%) negative predictive value for the absence of the three pathogens responsible for most acute otitis media in developed countries (Gehanno et al. (1996) Pediatr. Infect. Disease J., 15:329-332, which is incorporated by reference in its entirety herein). The method of the invention can be adapted to test nasopharyngeally derived samples for the presence, above a reference concentration, of the two other main AOM pathogens, Moraxella catarrhalis and non-typable Haemophilus influenzae. With respect to AOM clinical diagnosis in the developed world, it is highly desirable to combine into a single, convenient test device, a group of assays for rapidly ascertaining whether a subject is healthy (although possibly colonized) or diseased by the main pathogens of interest (S. pneumoniae, M. catarrhalis, and non-typable H. influenzae).

With respect to other infectious respiratory or non-respiratory diseases where the causative infectious organism can be at least potentially found in the nasopharyngeal area of the diseased person, it is also highly desirable to combine into a single test device, groups of assays for rapidly determining if a subject is healthy (although possibly colonized) or diseased by the infectious organisms that cause that disease. Thus, this invention encompasses assays and kits to test, individually or in combination, for pathogens that cause pneumonia, influenza and influenza-like illnesses, bronchitis, sinusitis, and conjunctivitis. In addition, this invention encompasses assays and kits to determine the presence of a pathogen that can cause an infectious disease of interest, or that can cause disease symptoms for which a diagnostic differential (for example, from non-pathogen-related chronic obstructive pulmonary disease) is necessary for appropriate therapy.

### EXAMPLES

### Example 1

### Rapid Detection of an Infectious Organism 1N Nasopharyngeal Samples

This example describes the disease-predictive value of detecting pneumococcal antigen in nasopharyngeal samples. A rapid pneumococcal antigen test was used to detect the presence or absence of S. pneumoniae in the nasopharynxes of children with or without acute otitis media.

Streptococcus pneumoniae is the leading bacterial cause of respiratory infections in children and of acute otitis media (AOM) in particular (Peter and Klein (1997) in: “Principles and Practice of Pediatric Infectious Diseases”, Long et al. (editors), Churchill Livingstone, New York, N.Y., pp. 828-835). Simultaneous cultures from nasopharynx and middle ear samples from patients with AOM demonstrate the same pathogen in a high proportion of cases (see, for example, Dickinson et al. (1988) J. Infect. Dis., 158:205-208; Faden et al. (1990) Pediatr. Infect. Dis. J., 9:623-626; Gehanno et al. (1996) Pediatr. Infect Dis. J., 15:329-332; Howie et al. (1971) Pediatr. Digest, 13:31-35; Loos et al. (1989) Infect. Immun., 57:2751-2757; and Schwartz et al. (1979) J. Am. Med. Assoc., 241:2170-2173, which are incorporated by reference in their entirety herein).

An in vitro rapid immunochromatographic assay (NOW® ICT Test for Streptococcus pneumoniae, Binax, Inc., Portland, Me.) is federally approved for the detection of pneumococcal soluble antigen in urine specimens from patients with symptoms of pneumonia. The binding agent used in the NOW® ICT Test is an antibody that is capable of specifically binding an epitope derived from the infectious organism (a single cell wall polysaccharide that is present on all clinical strains of S. pneumoniae) (see Miller et al. (1990) Arch. Otolaryngol. Head Neck Surg., 116:335-336; and Palv and Lehtinen (1987) Int. J. Pediatr. Otorhinolaryngol., 14:123-128, which are incorporated by reference in their entirety herein). Thus the NOW® ICT Test can detect all isolates of S. pneumoniae, unlike other available commercially immunochemistry-based kits, which use countercurrent immunoelectrophoresis or latex agglutination and are limited in their detection to the more common S. pneumoniae types. The NOW® ICT Test is less expensive and technically simpler than polymerase chain reaction (PCR) detection methods, and does not require specially trained technicians or sophisticated equipment. The NOW® ICT Test kit incorporates rabbit anti-S. pneumoniae antibody as the binding agent, adsorbed onto a nitrocellulose membrane. If pneumococcal antigen is in the specimen, an easily discernible pink-to-purple line appears within 15 minutes on the membrane. A control is included to ensure the validity of the test. For samples consisting of cerebrospinal fluid, the NOW® ICT Test gives a positive result when S. pneumoniae is present at a concentration greater than or equal to a reference concentration of 1×10⁴ colony-forming units per milliliter, with 100% overall detection when S. pneumoniae is present at a concentration greater than or equal to a reference concentration of 5×10⁴ colony-forming units per milliliter at expiration date of the test (NOW® Streptococcus pneumoniae Test Product Instructions, Binax, Inc., Portland, Me., which is incorporated by reference in its entirety herein).

One hundred thirty-eight subjects were enrolled at three sites after informed consent was obtained. The subjects were children below the age of 15 years, who were either healthy or clinically ill with acute otitis media. The subjects were enrolled without regard to sex or race. Children were excluded from the study if they had been treated with antibiotics within the past month.

The NOW® ICT Test was adapted for use in testing for pneumococcal antigen in nasopharyngeal samples from children (Faden et al. (2002) J. Clin. Microbiol., 40:4748-4749.). Nasopharyngeal samples were obtained with swabs (Mini-tip Culturettes, Becton Dickinson, Sparks, Md.). The same swab was used to collect the nasopharyngeal sample for antigen testing with the NOW® ICT Test and for culture to verify the presence or absence of S. pneumoniae in the nasopharyngeal sample. For antigen testing with the NOW® ICT Test, the nasopharyngeal swab samples were tested according to the directions in the kit.

The NOW® ICT Test for S. pneumoniae provides an immunochromatographic membrane assay device (see U.S. Pat. No. 5,877,028 and U.S. patent application Ser. No. 09/156,486, U.S. patent application Ser. No. 09/397,110, and U.S. patent application Ser. No. 09/518,165, which are incorporated by reference in their entirety herein) which includes a nitrocellulose membrane containing a rabbit anti-pneumococcal antigen antibody permanently immobilized by adsorption as a first stripe (“sample line”) and a control antibody permanently immobilized by adsorption as a second stripe (“control line”). The device also includes a conjugate pad (an inert fibrous support) containing a rabbit anti-pneumococcal antigen antibody and anti-species antibody, both of which are conjugated to visualizing gold particles and temporarily immobilized by drying onto the conjugate pad. The conjugate pad and the striped nitrocellulose membrane are combined into a test strip mounted on one side of a hinged, book-shaped device, which also contains a well to hold the swab sample on the side opposite to the test strip.

Briefly, the nasopharyngeal swab sample was inserted into well of the test device. A buffer, containing detergent and sodium azide, was added from a dropper bottle to the well, and the device closed, bringing the sample into contact with the test strip. Pneumococcal antigen that is present in the sample was specifically bound by the gold-conjugated rabbit anti-pneumococcal antigen antibody to form a complex. The resulting complex was captured by the rabbit anti-pneumococcal antigen antibody immobilized in the sample line of the test strip to form a visually detected signal (a pink-to-purple colored line) when sufficient complex is formed. The gold-conjugated anti-species antibody was captured by the control antibody immobilized in the control line of the test strip to also give a visually detected colored line. Test results were positive if a pink-to-purple colored line appeared on the sample line within 15 minutes or less, and negative if no pink-to-purple colored line appeared on the sample line in 15 minutes. The control line should have been visible for the assay to be valid.

For culture, the nasopharyngeal swab samples were cultured within 12 hours of collection on sheep blood agar and chocolate agar. The plates were incubated at 36° C. in a 5% carbon dioxide atmosphere for 18 to 24 hours. S. pneumoniae was identified by colonial morphology, Gram stain characteristics, optochin sensitivity, and bile solubility. Non-typable Haemophilus influenzae was identified by growth on chocolate agar, colonial morphology, Gram strain characteristics, a growth requirement for X and V factors, and failure to agglutinate with typing antisera. Moraxella catarrhalis was identified by colonial morphology, Gram stain characteristics, and a positive butyrate esterase test.

The one hundred thirty-eight subjects ranged in age from 4 to 168 months, with a median of 22.5 months. Seventy-two subjects were male, and 66 were female. Fifty-three children were classified as healthy, and 85 were classified as having acute otitis media (AOM). Nasopharyngeal cultures were collected from every subject. S. pneumoniae was recovered from 37% of the children. Healthy children were colonized less often with pathogens than were children with AOM (45.3% versus 87.1%, P<0.001). S. pneumoniae was recovered from 20.8% of healthy children and 47.1% of children with acute otitis media (P<0.01).

The sensitivity, specificity, positive predictive value, and negative predictive value of the NOW® ICT Test were calculated for the total population, and for the healthy and AOM subpopulations as well. Differences between the groups were assessed by chi square analysis. The NOW® ICT Test result was positive for 35.5% of the samples and negative for 64.5%. The NOW® ICT Test had an overall sensitivity, specificity, positive predictive value, and negative predictive value of 92.2, 97.7, 95.9, and 95.5%, respectively. The results for the healthy and AOM subpopulations were similar. Thus, a negative NOW® ICT S. pneumoniae Test result from a nasopharyngeal sample indicates the absence of S. pneumoniae in the sample, and therefore reliably predicts the absence of acute otitis media caused by S. pneumoniae in the subject.

### Example 2

### Reference Concentrations

In Example 1, the 20.8% of healthy subjects from whom S. pneumoniae was recovered by culture could be carriers, that is to say, healthy but colonized by S. pneumoniae. Such carriers can give a “false positive” result in terms of negative predictive value. The incidence of such “false positives” can be decreased by increasing the S. pneumoniae reference concentration for nasopharyngeal swab samples, preferably to from about 1×10⁴ colony-forming units per milliliter to about 5×10⁴ colony-forming units per milliliter; or 5×10⁴ colony-forming units per milliliter to about 5×10⁵ colony-forming units per milliliter; or from about 5×10⁵ colony-forming units per milliliter to about 5×10⁶ colony-forming units per milliliter, or from about 5×10⁶ colony-forming units per milliliter to about 5×10⁵ colony-forming units per milliliter, or from about 5×10⁷ colony-forming units per milliliter to about 5×1 0⁸ colony-forming units per milliliter. An acceptable reference concentration may be easily determined by methods as described above in Example 1, for example, by using the same rapid immunochromatographic device with the amounts of reagent, such as of the binding agent, adjusted appropriately.

A reference concentration for each infectious organism, type of nasopharyngeal sample, assay format, and detection method must be established by testing. Such reference concentrations can be in any range that is suitable for yielding an acceptable positive predictive value, a negative predictive value, or both, for a disease state of interest. Infectious organisms of interest include, but are not limited to:
(1) pathogens that can cause acute otitis media (non-typable Haemophilus influenzae and Moraxella catarrhalis, in addition to Streptococcus pneumoniae, as shown in Example 1);
(2) pathogens that can cause influenza-like illness (see, for example, Centers for Disease Control (2001) Morbidity Mortality Weekly Report, 50(44):984-986, which is incorporated by reference in its entirety herein), such as viruses (including, but not limited to, an influenza virus, a rhinovirus, a respiratory syncytial virus, an adenovirus, a parainfluenza virus, a coronavirus, and a metapneumovirus) and bacteria (including, but not limited to, Streptococcus pneumoniae, Chlamydia pneumoniae, and Mycoplasma pneumoniae);
(3) pathogens that can cause bacterial pneumonia (including, but not limited to Group A Streptococcus, Streptococcus pyogenes, Streptococcus pneumoniae, Klebsiella pneumoniae, Staphylococcus species, Haemophilus influenzae, Chlamydia pneumoniae, Mycoplasma pneumoniae, and Pseudomonas species), viral pneumonia (including, but not limited to, an influenza virus, a rhinovirus, a respiratory syncytial virus, an adenovirus, a parainfluenza virus, a coronavirus, a hantavirus, a cytomegalovirus, and a metapneumovirus), or fungal pneumonia (including, but not limited to, Histoplasma capsulatum, Coccidioides immitis, Blastomyces dermatitidis, Paracoccidioides brasiliensis, Candida species, Aspergillus species, Mucor species, Cryptococcus neoformans, and Pneumocystis carinii) (see, for example, Richards et al. (1994) Arch. Dis. Child., 71:254-255, which is incorporated by reference in its entirety herein);
(4) pathogens that can cause bronchitis, such as bacteria (including, but not limited to, Mycoplasma species such as Mycoplasma pneumoniae, Chlamydia pneumoniae, Bordatella pertussis, Group A Streptococcus, Streptococcus pyogenes, Streptococcus pneumoniae, Moraxella catarrhalis, Haemophilus influenzae, Haemophilus parainfluenzae, and Staphylococcus aureus) and viruses (including, but not limited to, an influenza virus, a rhinovirus, a respiratory syncytial virus, an adenovirus, a parainfluenza virus, a coronavirus, a hantavirus, a cytomegalovirus, and a metapneumovirus);
(5) pathogens that can cause sinusitis, such as bacteria (including, but not limited to, Streptococcus species such as Streptococcus pneumoniae, Haemophilus influenzae, Staphylococcus species such as Staphylococcus aureus, Moraxella catarrhalis, and Neisseria species) or fungi; and
(6) pathogens that can cause conjunctivitis, such as bacteria (including, but not limited to, Group A Streptococcus, Streptococcus pyogenes, Streptococcus pneumoniae, Staphylococcus epidermidis, Streptococcus Aureas, Haemophilus species, Haemophilus influenzae, Neisseria species such as Neisseria meningitidis and Neisseria gonorrhoeae, Moraxella lacunata, and Chlamydia species) or viruses.

All headings are for the convenience of the reader and should not be used to limit the meaning of the text that follows the heading, unless so specified. Various changes and departures may be made to the present invention without departing from the spirit and scope thereof. Accordingly, it is not intended that the invention be limited to that specifically described in the specification or as illustrated in the drawings, but only as set forth in the claims.

1. A method of rapidly determining the disease state of a subject, wherein said disease is caused by an infectious organism, comprising the steps of:
a) providing a nasopharyngeally derived sample from said subject;
b) contacting a binding agent directly to said sample, wherein said binding agent is capable of specifically binding to an epitope derived from said infectious organism;
c) allowing said binding agent to specifically bind to and form a complex with said epitope derived from said infectious organism present in said sample; and
d) detecting said complex, wherein said detection is positive if concentration of said infectious organism in said sample is greater than or equal to a reference concentration, and said detection is negative if concentration of said infectious organism in said sample is less than said reference concentration.

2. The method of claim 1, wherein positive detection indicates that the subject is diseased.

3. The method of claim 1, wherein negative detection indicates that the subject is not diseased.

4. The method of claim 2, wherein said positive detection is optionally quantitative.

5. The method of claim 1, wherein said infectious organism is selected from the group consisting of pathological bacteria, pathological viruses, and pathological eukaryotes.

6. The method of claim 1, wherein said disease is acute otitis media.

7. The method of claim 6, wherein said infectious organism is non-typable Haemophilus influenzae.

8. The method of claim 6, wherein said infectious organism is Streptococcus pneumoniae.

9. The method of claim 6, wherein said infectious organism is Moraxella catarrhalis.

10. The method of claim 6, wherein said infectious organism is a Group A Streptococcus.

11. The method of claim 10, wherein said Group A Streptococcus is Streptococcus pyogenes.

12. The method of claim 1, wherein said disease is a respiratory tract infection.

13. The method of claim 12, wherein said respiratory tract infection is an influenza-like illness.

14. The method of claim 13, wherein said infectious organism is a virus.

15. The method of claim 14, wherein said virus is an influenza virus.

16. The method of claim 14, wherein said virus is a rhinovirus.

17. The method of claim 14, wherein said virus is a respiratory syncytial virus.

18. The method of claim 14, wherein said virus is an adenovirus.

19. The method of claim 14, wherein said virus is a parainfluenza virus.

20. The method of claim 14, wherein said virus is a coronavirus.

21. The method of claim 14, wherein said virus is a metapneumovirus.

22. The method of claim 13, wherein said infectious organism is a bacterium.

23. The method of claim 22, wherein said bacterium is Streptococcus pneumoniae.

24. The method of claim 22, wherein said bacterium is Chlamydia pneumoniae.

25. The method of claim 22, wherein said bacterium is Mycoplasma pneumoniae.

26. The method of claim 12, wherein said respiratory tract infection is pneumonia.

27. The method of claim 26, wherein said pneumonia is a bacterial pneumonia.

28. The method of claim 27, wherein said infectious organism is a Group A Streptococcus pneumoniae.

29. The method of claim 28, wherein said Group A Streptococcus is Streptococcus pyogenes.

30. The method of claim 27, wherein said infectious organism is Streptococcus pneumoniae.

31. The method of claim 27, wherein said infectious organism is Klebsiella pneumoniae.

32. The method of claim 27, wherein said infectious organism is a Staphylococcus species.

33. The method of claim 27, wherein said infectious organism is Haemophilus influenzae.

34. The method of claim 27, wherein said infectious organism is Chlamydia pneumoniae.

35. The method of claim 27, wherein said infectious organism is Mycoplasma pneumoniae.

36. The method of claim 27, wherein said infectious organism is a Pseudomonas species.

37. The method of claim 26, wherein said pneumonia is a viral pneumonia.

38. The method of claim 37, wherein said infectious organism is a respiratory syncytial virus.

39. The method of claim 37, wherein said infectious organism is a rhinovirus.

40. The method of claim 37, wherein said infectious organism is an adenovirus.

41. The method of claim 37, wherein said infectious organism is an influenza virus.

42. The method of claim 37, wherein said infectious organism is a parainfluenza virus.

43. The method of claim 37, wherein said infectious organism is a coronavirus.

44. The method of claim 37, wherein said infectious organism is a hantavirus.

45. The method of claim 37, wherein said infectious organism is a cytomegalovirus.

46. The method of claim 37, wherein said infectious organism is a metapneumovirus.

47. The method of claim 26, wherein said pneumonia is a fungal pneumonia.

48. The method of claim 47, wherein said infectious organism is Histoplasma capsulatum.

49. The method of claim 47, wherein said infectious organism is Coccidioides immitis.

50. The method of claim 47, wherein said infectious organism is Blastomyces dermatitidis.

51. The method of claim 47, wherein said infectious organism is Paracoccidioides brasiliensis.

52. The method of claim 47, wherein said infectious organism is a Candida species.

53. The method of claim 47, wherein said infectious organism is an Aspergillus species.

54. The method of claim 47, wherein said infectious organism is a Mucor species.

55. The method of claim 47, wherein said infectious organism is Cryptococcus neoformans.

56. The method of claim 47, wherein said infectious organism is Pneumocystis carinii.

57. The method of claim 12, wherein said respiratory tract infection is bronchitis.

58. The method of claim 57, wherein said infectious organism is a bacterium.

59. The method of claim 58, wherein said bacterium is a Mycoplasma species.

60. The method of claim 58, wherein said bacterium is Mycoplasma pneumoniae.

61. The method of claim 58, wherein said bacterium is Chlamydia pneumoniae.

62. The method of claim 58, wherein said bacterium is Bordatella pertussis.

63. The method of claim 58, wherein said bacterium is a Group A Streptococcus.

64. The method of claim 63, wherein said Group A Streptococcus is Streptococcus pyogenes.

65. The method of claim 58, wherein said bacterium is Streptococcus pneumoniae.

66. The method of claim 58, wherein said bacterium is Moraxella catarrhalis.

67. The method of claim 58, wherein said bacterium is Haemophilus influenzae.

68. The method of claim 58, wherein said bacterium is Haemophilus parainfluenzae.

69. The method of claim 58, wherein said bacterium is Staphylococcus aureus.

70. The method of claim 57, wherein said infectious organism is a virus.

71. The method of claim 70, wherein said virus is an influenza virus.

72. The method of claim 70, wherein said virus is a parainfluenza virus.

73. The method of claim 70, wherein said virus is an adenovirus.

74. The method of claim 70, wherein said virus is a rhinovirus.

75. The method of claim 70, wherein said virus is a respiratory syncytial virus.

76. The method of claim 70, wherein said virus is a coronavirus.

77. The method of claim 70, wherein said virus is a hantavirus.

78. The method of claim 70, wherein said virus is a metapneumovirus.

79. The method of claim 12, wherein said respiratory tract infection is sinusitis.

80. The method of claim 79, wherein said infectious organism is a bacterium.

81. The method of claim 80, wherein said bacterium is a Streptococcus species.

82. The method of claim 80, wherein said bacterium is Streptococcus pneumoniae.

83. The method of claim 80, wherein said bacterium is Haemophilus influenzae.

84. The method of claim 80, wherein said bacterium is a Staphylococcus species.

85. The method of claim 80, wherein said bacterium is Staphylococcus aureus.

86. The method of claim 80, wherein said bacterium is a Neisseria species.

87. The method of claim 79, wherein said infectious organism is a fungus.

88. The method of claim 1, wherein said disease is conjunctivitis.

89. The method of claim 88, wherein said infectious organism is a bacterium.

90. The method of claim 89, wherein said bacterium is a Group A Streptococcus.

91. The method of claim 90, wherein said Group A Streptococcus is Streptococcus pyogenes.

92. The method of claim 89, wherein said bacterium is Streptococcus pneumoniae.

93. The method of claim 89, wherein said bacterium is Staphylococcus epidermidis.

94. The method of claim 89, wherein said bacterium is a Haemophilus species.

95. The method of claim 89, wherein said bacterium is Haemophilus influenzae.

96. The method of claim 89, wherein said bacterium is Neisseria meningitidis.

97. The method of claim 89, wherein said bacterium is Neisseria gonorrhoeae.

98. The method of claim 89, wherein said bacterium is Moraxella lacunata.

99. The method of claim 89, wherein said bacterium is a Chlamydia species.

100. The method of claim 88, wherein said infectious organism is a virus.

101. The method of claim 1, wherein said nasopharyngeally derived sample is selected from the group consisting of a nasopharyngeal swab, a nasopharyngeal wash, nasopharyngeal discharge, nasopharyngeal aspirate, nasal swab, nasal wash, nasal discharge, nasal aspirate, and a combination thereof.

102. The method of claim 1, wherein said method further comprises simultaneous or parallel detection of more than one infectious organism.

103. The method of claim 1, wherein said epitope is modified.

104. The method of claim 1, wherein said binding agent comprises an antibody or antibody fragment.

105. The method of claim 1, wherein said binding agent comprises a functional group or a detectable label.

106. The method of claim 1, wherein said binding agent is used in more than one form.

107. The method of claim 1, wherein said binding agent is further capable of binding to a mimotope that mimics said epitope derived from said infectious organism.

108. A kit for performing the method of claim 1.
